(19) United States
(12) Patent Application Publication
(10) Pub. No.: US 2008/0226702 A1
(43) Pub. Date: Sep. 18, 2008

(54) Transdermal Delivery Form Disposal Systems and Methods

(75) Inventor: Alan Philip Goldberg, Wilmington, DE (US)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Endo Pharmaceuticals, Inc.

(21) Appl. No.: 12/048,771

(22) Filed: Mar. 14, 2008

Related U.S. Application Data
(60) Provisional application No. 60/918,650, filed on Mar. 16, 2007.

(51) Int. Cl. A61F 13/02; A61K 31/4535; A61K 31/445

(52) U.S. Cl. 424/449; 514/326; 514/329

(57) ABSTRACT

The extraction and use of residual opioids from transdermal dosage forms can be reduced by placement and fixation of used dosage forms onto a surface. The used dosage form can be fixed to the surface such that it cannot be removed without at least partially destroying the matrix containing the opioid, or such that the matrix containing the opioid is rendered at least partially inaccessible to opioid extraction methods. The disposal system for one or more opioid-containing transdermal delivery forms comprises at least one disposal surface for receiving the transdermal delivery form. The at least one disposal surface can comprise one or more structures for fixing a transdermal delivery form to the at least one surface. Alternatively, the transdermal delivery form comprises one or more structures for permanently fixing the transdermal delivery form to the surface. The disposal system can also be used for preventing or reducing diversion of opioids from transdermal delivery forms.

### CROSS REFERENCE TO RELATED APPLICATION

This application claims priority from U.S. Ser. No. 60/918,650, filed Mar. 16, 2007, the contents of which are incorporated herein by reference.

### TECHNICAL FIELD

This invention relates to systems for disposing of transdermal drug delivery forms which can contain a controlled substance, to methods of making and using such systems, and to methods of reducing or preventing diversion of controlled substances.

### BACKGROUND

Opioid compounds have long been known for their powerful analgesic properties. While highly effective at controlling pain, opioids can be extremely addictive. The physical and psychological addiction produced by the opioids can be so strong that addicts will repeatedly self-administer the drugs to the point of physical harm or death. To satisfy their compulsion, opioid addicts can be driven to obtain drugs from a variety of licit and illicit sources. Opioids obtained from the “street” are of questionable quality. Therefore, prescription pharmaceutical opioids can be particularly attractive as a drug source for opioid addicts because of the high purity and dependable dosage.

The development of different dosage forms and strengths for prescription opioids has increased the availability of such drugs to addicts. For example, addicts can obtain unused prescription opioids from patients who have been legally provided with the drugs. Opioid dosage forms which been used by the patient, but which contain residual amounts of the drug, can also be a significant source of opioids for addicts.

Transdermal opioid dosage forms, such as the DURAGESIC® and TRANSDUR® patches, can have significant drug load overages to assure sufficient flux rates of opioid through the skin. Transdermal opioid dosage forms are therefore being targeted by addicts as a supply of opioids for abuse. Used patches containing residual drug can be collected from hospital or home refuse, and the opioids contained in these patches can be extracted and illicitly supplied to addicts. Addicts typically administer these extracted opioids orally, intranasally or parenterally.

Thus, physician and hospitals are often faced with a choice of keeping and prescribing potent opioid analgesics in a transdermal delivery form, or using a less effective (but less addictive) analgesic to treat patients. To mitigate this choice, health care providers often will only provide a few days' supply of transdermal delivery forms to patients for use outside the hospital setting. This is inconvenient for those who need longer term administration of the drug. However, due to the number of patients treated, hospitals and other health care facilities often dispose of sizeable quantities of used transdermal opioid delivery forms on a daily basis. Private citizens using transdermal opioids by prescription at home risk having their refuse searched for the used transdermal delivery forms.

In order to reduce the potential that residual opioids from transdermal delivery forms can be extracted and abused, such delivery forms can contain substances such as opioid antagonists or irritants which reduce the oral, intranasal or parenteral effectiveness of opioids. Such opioid antagonists and irritants will be co-extracted from the transdermal delivery form with the opioids. For example, naloxone is a powerful antagonist of the opioid receptor, and reduces the “high” experienced by addicts when taken parenterally with an opioid. Oral or intranasal administration of opioids by addicts can also be curtailed by including co-extractable irritants, such as capsaicin, in the transdermal delivery form.

Transdermal delivery forms may contain other controlled substances that can create physical or psychological addiction. Such delivery forms may also be sought by addicts or those wishing to supply addicts.

However, the inclusion of antagonists or irritants in transdermal delivery forms may not be sufficient to prevent many addicts from extracting and administering residual controlled substances, such as opioids. The antagonists or irritants can also be separated from the extracted controlled substance or tolerated by a determined individual. Thus, there is a need for a disposal system for transdermal dosage forms containing controlled substances, in particular opioids, which further frustrates attempts to recover and use the residual controlled substance. Desirably, such a system can also track or otherwise account for each used transdermal delivery form.

### SUMMARY

The extraction and use of residual controlled substances from transdermal dosage delivery forms can be reduced by placement and fixation of used dosage forms onto a disposal surface. The used delivery form is fixed to the surface such that it cannot be removed without at least partially destroying the matrix containing the controlled substance, or such that the matrix containing the controlled substance is rendered at least partially inaccessible to extraction methods.

Thus, a disposal system for one or more used transdermal delivery forms containing controlled substances is provided, comprising at least one disposal surface for receiving the transdermal delivery form. The at least one disposal surface can comprise one or more structures for fixing a transdermal delivery form to the at least one disposal surface. Alternatively, the one or more used transdermal delivery forms comprise one or more structures for fixing the transdermal delivery forms to the surface.

A method of disposing of one or more used transdermal delivery forms containing controlled substances is also provided. The method comprises providing at least one disposal surface for receiving the one or more used transdermal delivery forms, placing the one or more transdermal delivery forms on the at least one disposal surface, and fixing the used transdermal delivery forms to the disposal surface such that they cannot be removed without at least partially destroying the matrix containing the controlled substance, or such that the matrix containing the controlled substance is rendered at least partially inaccessible to extraction methods. Either the at least one disposal surface or the one or more transdermal delivery forms, or both, can comprise at least one structure for fixing the transdermal delivery forms to the disposal surface.

The disposal system described herein can also be used to reduce or prevent diversion of controlled substances contained in one or more used transdermal delivery forms.

A method of producing a disposal system for one or more used transdermal delivery forms containing controlled substances is also provided. The method comprises the provision of one or more used transdermal delivery forms, and the formation of at least one surface for receiving the used transdermal delivery form. The one or more used transdermal delivery forms or the at least one disposal surface, or both, can comprise one or more structures for fixing a used transdermal delivery form to the at least one surface.

### BRIEF DESCRIPTION OF THE FIGURES

For the purpose of illustrating representative aspects of the invention, there are shown in the figures forms which are exemplary, it being understood that this disclosure is not limited to the precise arrangements and instrumentalities shown.

FIG. 1 is a plan view of an exemplary transdermal delivery form, showing the surface intended to contact the skin.

FIG. 2 is a schematic of an exemplary disposal system, in top plan view.

FIG. 3 is a schematic of a further exemplary disposal system, in top plan view.

FIG. 4 is a schematic of a further exemplary disposal system, in top plan view.

FIG. 5 is a schematic of a further exemplary disposal system, in top plan view.

### DETAILED DESCRIPTION

Transdermal drug delivery devices, sometimes referred to herein as “transdermal drug delivery dosage forms,” “transdermal delivery forms” or “delivery forms,” typically comprise a carrier (such as, for example, a liquid, gel, or solid matrix, or a pressure sensitive adhesive) into which an active substance to be delivered is incorporated. Because the skin presents a substantial barrier to ingress of foreign substances, it is often desirable or necessary to incorporate excipients into the carrier that enhance the rate at which the active substance passes through the skin. Transdermal delivery forms known in the art include reservoir-type devices with membranes that control the rate of drug and/or skin penetration enhancer delivery to the skin. There are also “single layer” devices involving a dispersion or solution of drug and excipients in an adhesive matrix, and more complex multilaminate devices involving several distinct layers; e.g., layers for containing drug, for containing skin penetration enhancer, for controlling the rate of release of the drug and skin penetration enhancer, for attaching the device to the skin and the like.

Reservoir-type transdermal delivery forms contain a drug in a fluid or gel matrix carrier in the reservoir. In use, the drug diffuses out of the matrix and across a membrane to provide controlled release through the skin. “Single layer” transdermal delivery forms are those in which the drug is directly dispersed or dissolved in a single adhesive layer, which usually comprises a pressure sensitive adhesive matrix. Such delivery forms typically include an inert, impervious backing layer, a pressure sensitive adhesive layer containing the drug and optionally selected excipients, and a release liner that is peeled off and discarded before applying the delivery form to the skin. Examples of suitable pressure sensitive adhesives include polysiloxanes, polyacrylates, polyisobutylene, and the like. These pressure sensitive adhesive polymers are hydrophobic and are typically formed on the backing layer as solutions of polymer dissolved in organic solvents. The drug and selected excipients, if any, are directly incorporated into the organic-solvent-based pressure sensitive adhesive solution, mixed, cast as a thin film, and dried to evaporate the solvents, leaving a dried adhesive matrix film containing the drug and excipients. These and other transdermal delivery forms, especially those for opioids, and methods of their manufacture and use are known to those of ordinary skill in the art, for example, as described in U.S. Pat. Nos. 4,626,539; 5,762,952; 5,948,433; 5,985,317; 6,110,488; and 6,89,3,655, the entire disclosures of which are herein incorporated by reference.

In particular, delivery forms that can be used with the disposal system and methods discussed herein include transdermal patches marketed under the trademark DURAGESIC® (Janssen Pharmaceutica, Titusville, N.J.). DURAGESIC® is a rectangular transparent unit comprising a protective liner and four functional layers. Proceeding from the outer surface toward the surface adhering to skin, these layers are: 1) a backing layer of polyester film; 2) a drug reservoir of fentanyl and alcohol USP gelled with hydroxyethyl cellulose; 3) an ethylene-vinyl acetate copolymer membrane that controls the rate of fentanyl delivery to the skin surface; and 4) a fentanyl containing silicone adhesive.

Another delivery system that can be used with the disposal system and methods discussed herein includes the transdermal patch for delivery of sufentanil, called TRANSDUR®, which is being developed by Endo Pharmaceuticals (Chadds Ford, Pa.). This patch is intended to provide continuous delivery of sufentanil for up to seven days from a single application, as compared to the three days of relief provided by currently available opioid patches such as DUBAGESIC®.

The disposal system discussed herein is concerned with transdermal delivery forms which contain controlled substances, in particular opstance, or by those wishing to supply addicts with the controlled substance. As used herein, “diversion” or “diverted” with respect to a controlled substance means that the controlled substance has been acquired by an individual other than for whom it was prescribed or intended. A controlled substance that has been diverted is typically provided to an addict. Addicts will often self-administer a controlled substance, especially an opioid, to the point of physical harm or death. The reduction or prevention of diversion of controlled substances is therefore a specific application of the disposal system.

It is understood that the disposal system and methods discussed herein can be used with transdermal dosage forms that contain any controlled substance. As used herein, a “controlled substance” is any pharmacologically active substance which can produce physical or psychological addiction, whether or not it is illegal in a given jurisdiction to possess the substance without a license or prescription. Examples of controlled substances include opioids, benzodiazepines such as Valium, and NMDA-antagonists such as ketamine. However, for ease of illustration, the disposal system and methods will be generally discussed below in terms of transdermal dosage forms that contain opioids. Opioids which can be contained in transdermal dosage forms include, but are not limited to, alfentanil, buprenorphine, butorphanol, codeine, dezocine, dihydrocodeine, fentanyl and fentanyl congeners (e.g., sufentanil, alfentanil, lofentanil, carfentanil, remifentanil, trefentanil, and mirfentanil), hydrocodone, hydromorphone, levorphanol, meperidine (pethidine), methadone, morphine, nalbuphine, oxycodone, oxymorphone, pentazocine, propiram, propoxyphene, tilidine, tramadol, the pharmaceutically acceptable acid addition salts thereof, and any combinations of these.

The transdermal delivery forms useful with the disposal system and methods may further include one or more other active ingredients that may be conventionally employed in analgesic combination products. Such conventional ingredients include, but are not limited to, aspirin or other salicylates, acetaminophen, phenylpropanolamine, phenylephrine, chlorpheniramine, caffeine and guaifenesin. Other conventional ingredients that may be included in the transdermal delivery forms are described, for example, in the Physicians' Desk Reference, 1999, the disclosure of which are hereby incorporated herein by reference in its entirety.

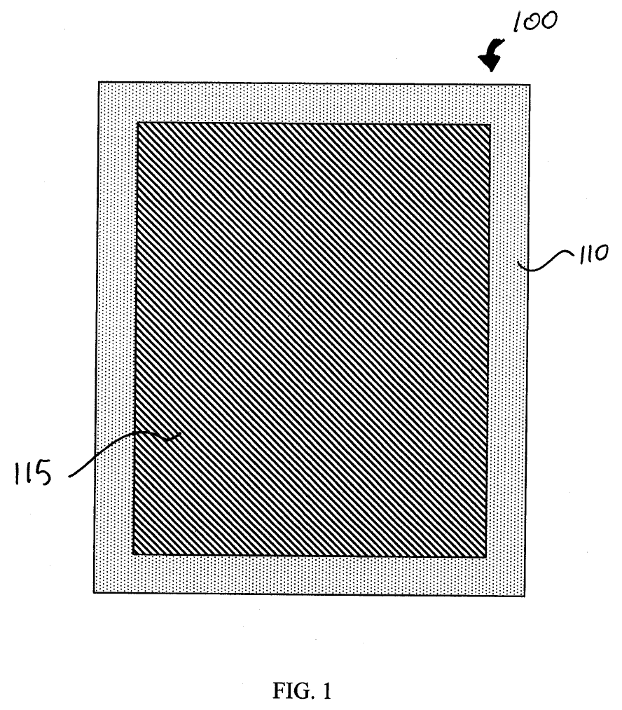

The transdermal delivery forms can comprise an adhesive which is disposed on part or all of the surface intended to contact the skin. For example, certain transdermal delivery forms can have adhesive disposed on substantially the entire surface intended to contact the skin. Other transdermal delivery forms can have adhesive disposed around substantially the entire perimeter of the surface intended to contact the skin (see FIG. 1). As shown in FIG. 1, transdermal delivery form 100 has an adhesive 110 which surrounds a central area 115 (such as a reservoir) which contains the opioid. The adhesive is primarily intended to secure the delivery form to the skin. However, in the disposal system and methods, the adhesive on the delivery system can also function to fix it to a surface provided in the disposal system which is constructed to receive a used delivery form.

The disposal system thus comprises at least one surface for receiving one or more used transdermal delivery forms. As used herein, a “used” transdermal delivery form includes one that contains residual opioid whether or not it has been administered to a patient. Hence, a “used” transdermal delivery form includes one that has not been administered to a patient, but which is to be discarded. For example, a transdermal patch which has expired or is damaged, and is thus unsuitable for patient use.

The at least one surface of the disposal system (sometimes called a “disposal surface”) can comprise one or more defined or undefined areas for receiving a used transdermal delivery form. In certain aspects, the disposal surface comprises one or more such areas which are defined; e.g., by lines, colorations or other suitable markings. The defined areas can also be marked with numbers, letters, alphanumeric codes or the like, for the purpose of tracking the transdermal delivery forms which have been fixed thereto. For example, the defined areas on the disposal surface can be marked to correspond to a given set of transdermal delivery forms. Thus, transdermal delivery form number “1A” can be placed in defined area “1A” of the disposal surface. In this way, a healthcare provider can ensure that all used transdermal delivery forms have been accounted for.

The disposal surface can comprise any suitable rigid or flexible material that can be formed into a generally smooth, flattened shape that can receive used transdermal delivery forms. For example, heavy gauge paper or cardboard, woods, metals, plastics, rubbers or synthetic resins, as are known in the art, can be used. Suitable materials include, but are not limited to, thermoplastic materials which can be fabricated by injection molding (or similar techniques), such as acrylonitrile-butadiene-styrene terpolymer (ABS); ionomer resin; ethylene vinyl acetate (EVA); thermo plastic styrenics (TPS); melt processible rubber (MPR); thermo plastic vulcanate (TPV); thermo plastic olefin (TPO); thermo plastic ester elastomer (TPEE); thermo plastic elastomer (TPE) such as thermo plastic polyurethane (TPU); thermoplastic rubber (TPR); polypropylene (PP), polyethylene terephthalate (PET), polyvinyl chloride (PVC); acrylonitrile-butadiene-styrene terpolymer (ABS); a polycarbonate and acrylonitrile-butadiene-styrene terpolymer blend (PC/ABS); flexible plastic such as polystyrene sheet or polymethylmethacrylate (PMMA, marketed as “PERSPEX” by ICI Acrylics, Inc.); other acrylics; metal (e.g., stainless steel, aluminum, copper); wood; or any combination thereof. Other suitable materials and forming methods will be apparent to those skilled in the art.

The disposal surface can have any suitable shape and dimensions (i.e., length, height and thickness) which allow the placement and fixation of one or more used transdermal delivery systems on at least a portion of the surface. The disposal surface can conveniently be shaped and dimensioned to receive a maximum number of used transdermal forms in a given area, but also be easily stored. For example, the disposal surface can be generally square or rectangular shaped, and can have a length and height of standard US letter-size or European A4 size paper. The disposal surface can have a thickness of about 0.5 mm to about 10 mm, for example about 1 mm to about 5 mm, or about 2 mm to about 4 mm. Other shapes and greater or lesser dimensions are also contemplated for the disposal surface.

The disposal system also comprises one or more structures for fixing a transdermal delivery form to the at least one disposal surface. These structures can be located on or attached to the disposal surface, or can be located or attached to the transdermal delivery form, or both. It is thus understood that one or more transdermal delivery forms can comprise part of the disposal system.

The one or more structures for fixing a transdermal delivery form to the at least one disposal surface can comprise any suitable element or combination of elements which serve to secure the delivery form to the disposal surface. For example, the structure can comprise one or more adhesives disposed on the delivery form and/or disposal surface. In addition to an adhesive, or as an alternative to an adhesive, the transdermal delivery form can be fixed to the disposal surface with a fastening device, for example, clamps, ties, brackets, staples, stitches or other suitable devices.

The adhesive can comprise a pressure sensitive adhesive as is known in the art. Pressure sensitive adhesive, including those described in Satas et al., Handbook of Pressure Sensitive Adhesives, 2d ed. 1989, the entire disclosure of which is herein incorporated by reference, can be used. Suitable classes of suitable pressure sensitive adhesives include, for example, acrylics, natural and synthetic rubbers, ethylene vinyl acetate, poly(alpha-olefins), vinyl ethers, silicones and combinations thereof. The adhesives may be in the form of copolymers, bicontinuous adhesives, hydrogels, latex emulsions, macromers, and block copolymers. Suitable block copolymers are commercially available from Shell Oil Company (Houston, Tex.) under the trade designation KRATON™.

Suitable acrylic adhesives are disclosed, for example, in U.S. Pat. Nos. 5,986,011; 5,637,646 and 5,753,768, the entire disclosures of which are herein incorporated by reference. A suitable class of acrylate pressure sensitive adhesives comprise the reaction product of at least one alkyl acrylate with at least one reinforcing comonomer. Suitable alkyl acrylates are those having a homopolymer glass transition temperature below about −10° C. and include, for example, n-butyl acrylate, 2-ethylhexylacrylate, isoctylacrylate, isononyl acrylate, ethylene monoacrylate, octadecyl acrylate and the like. Suitable reinforcing comonomers such as, for example, acrylic acid, itaconic acid, isobornyl acrylate, N,N-dimethylacrylamide, N-vinyl caprolactam, N-vinyl pyrrolidone, and the like.

An example of a suitable styrene/isoprene/styrene block copolymer pressure sensitive adhesives includes PL915M, which is commercially available from SIA Adhesives, Inc. (Akron, Ohio). This adhesive will produce an essentially permanent bond upon application of heat or pressure, or after an appreciable passage of time. Examples of other suitable adhesives include styrene/butadiene/styrene pressure sensitive adhesives, such as Products 8706, 8707, 8709 and 1191 commercially available from Avery Dennison, Fasson Films Div. (Painesville, Ohio), acrylic, butadiene-acrylonitrile, butyl rubber, natural rubber, silicone, polychloroprene, polyvinyl acetate, polyvinyl ether, polyurethanes or other synthetic rubber or resin systems as are known in the art, for example as described in U.S. Pat. Nos. 4,820,746 and 6,426,130 the entire disclosures of which are herein incorporated by reference.

The adhesive can also comprise a non-pressure sensitive adhesive as is known in the art. Any non-pressure sensitive adhesive can be used. Suitable non-pressure sensitive adhesive matrix materials include polymers comprising poly(meth)acrylate, polyvinylpyrrolidone, ethylcellulose, hydroxypropylcellulose, hydroxypropylmethyl-cellulosephthalate, polyvinylalcohol or copolymers thereof with vinyllaurate or maleic acid, vinylacetate or copolymers thereof with vinyllaurate or maleic acid, polyvinylether, butylrubber, polycaprolactam and combinations thereof.

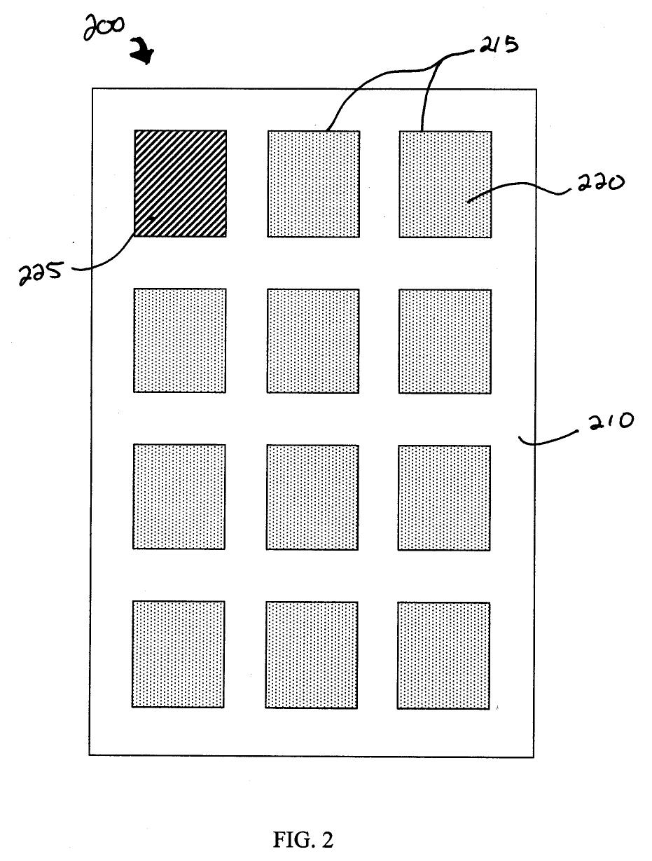

An exemplary disposal system 200 is generally shown in FIG. 2. The system comprises disposal surface 210. In the structure shown in FIG. 2, the surface is marked with a plurality of areas 215 which are each sized to receive a single used transdermal delivery form. Adhesive 220 is disposed on areas 215, and a used transdermal delivery form can be fixed to the surface by placing it substantially within an area 215 so that the transdermal delivery form surface that had been in contact with the skin is now in contact with adhesive 220. As described above, the transdermal delivery form may have adhesive on part or all of its skin-facing surface. Alternatively, the transdermal delivery system has no adhesive. The interaction between adhesive 220 and the transdermal delivery form (and adhesive on the delivery form, if any) is sufficient to fix the transdermal delivery form to surface 210. A transdermal delivery form 225 is shown fixed to an area 215 of surface 210. It is understood that the adhesive need not be limited to areas 215, but can be provided on substantially the entire disposal surface 210.

The structure for fixing the transdermal delivery form to the disposal surface can also comprise a flexible or rigid covering layer. The covering layer can be arranged to cover some or all of a disposal surface to which at least one transdermal delivery form has been placed. The covering layer can be attached to or can be separate from the disposal layer. For example, the covering layer can be attached to the disposal layer at a single common edge, which allows the covering layer to be folded back away from the disposal layer for placement of used transdermal delivery forms. Once one or more delivery forms have been placed on the disposal surface, the covering layer can be brought into contact with the disposal surface and secured thereto. Securing the covering layer to the disposal surface thus fixes the delivery devices in place. The covering layer, disposal surface, or both can comprise an adhesive to secure the covering layer and disposal surface together. Alternatively, the covering layer and disposal surface can be secured together by a fastening device such as clamps, ties, brackets, staples, stitches or other suitable devices. The covering layer can be fabricated using the same techniques and materials described above for the disposal surface.

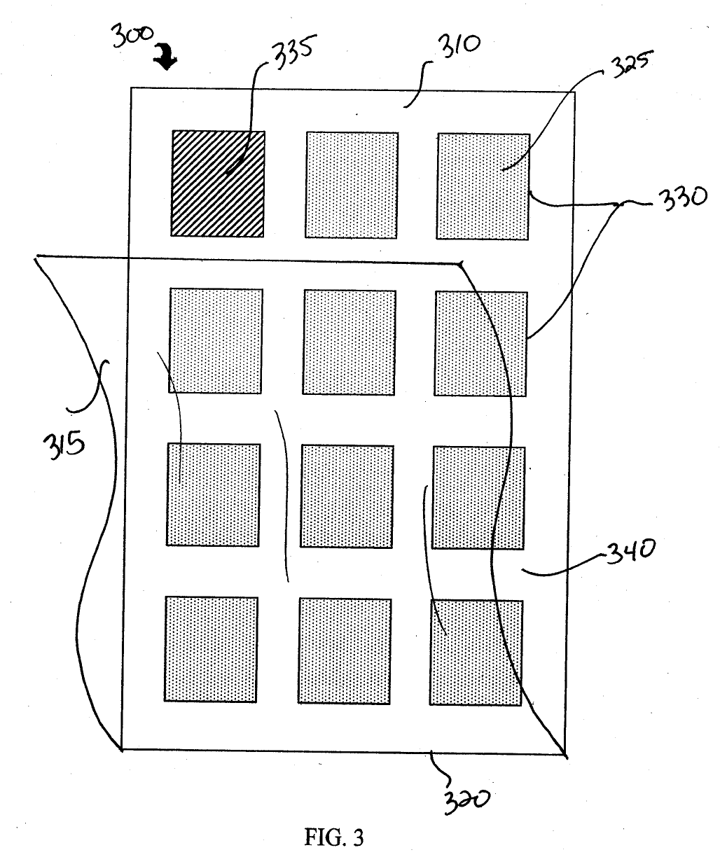

Another exemplary disposal system 300 is generally shown in FIG. 3. The system comprises disposal surface 310 and covering layer 315, which is flexibly attached to the disposal surface at edge 320. Adhesive 325 is disposed on areas 330, and a used transdermal delivery form can be fixed to the surface by placing it substantially within an area 330 so that the transdermal delivery form surface that had been in contact with the skin is now in contact with adhesive 325. As described above, the transdermal delivery form may have adhesive on part or all of its skin-facing surface. Alternatively, the transdermal delivery system has no adhesive. The interaction between adhesive 325 and the transdermal delivery form (and adhesive on the delivery form, if any) is sufficient to fix the transdermal delivery form to surface 310. A transdermal delivery form 335 is shown fixed to an area 330 of surface 310. An adhesive, which may be the same or different from adhesive 325, is disposed on some or all of the portion 340 of the disposal surface 310 which is between and around areas 330. Once one or more transdermal delivery forms have been placed on the disposal surface 310, the covering layer 315 can be positioned over and contacted with the disposal surface, such that the adhesive on portion 340 secures the covering layer in place.

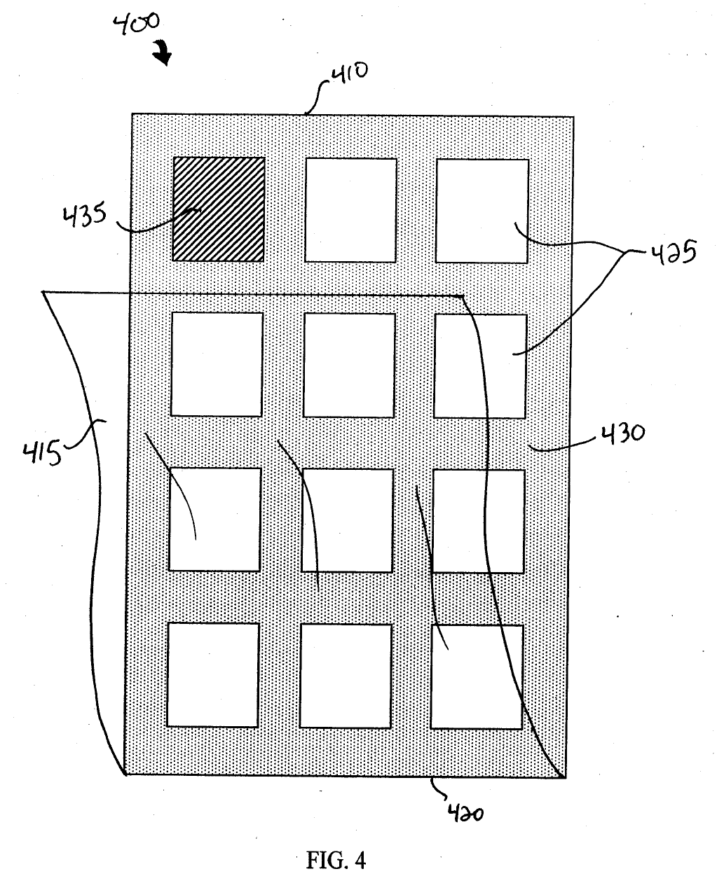

A further exemplary disposal system 400 is shown generally in FIG. 4. The system comprises disposal surface 410 and covering layer 415, which is flexibly attached to the disposal surface at edge 420. Areas 425 are provided for placement of used transdermal delivery forms. However, adhesive 430 is disposed on some or all of the portion of the disposal surface 410 which is between and around areas 425, and areas 425 have substantially no adhesive. As described above, the transdermal delivery form may have adhesive on part or all of its skin-facing surface. Alternatively, the transdermal delivery system has no adhesive. A transdermal delivery form 435 is shown placed in an area 425 of disposal surface 410. Once one or more transdermal delivery forms have been placed on the disposal surface 410, the covering layer 415 can be positioned over and contacted with the disposal surface, such that the adhesive 430 secures the covering layer 415 in place.

The covering layer can be substantially co-extensive with the disposal surface, can be larger than the disposal surface, or can only partially cover the disposal surface. The covering layer can also be any shape suitable to at least partially cover a used transdermal form which has been placed on the disposal surface.

In certain aspects, the covering layer covers at least part of the transdermal delivery forms which have been placed on the disposal surface. The covering layer also does not need to be continuous; rather, the covering layer can comprise a plurality of parts (for example strips), each of which partially or substantially completely, covers different transdermal delivery forms which have been placed on the disposal surface. The covering layer can also be any suitable thickness which allows the used transdermal forms to be fixed to the disposal layer. For example, the covering layer can have a thickness of about 0.1 mm to about 1 mm, about 0.15 mm to about 0.85 mm, or about 0.3 mm to about 0.5 mm. Greater or lesser thicknesses for the covering layer are also contemplated.

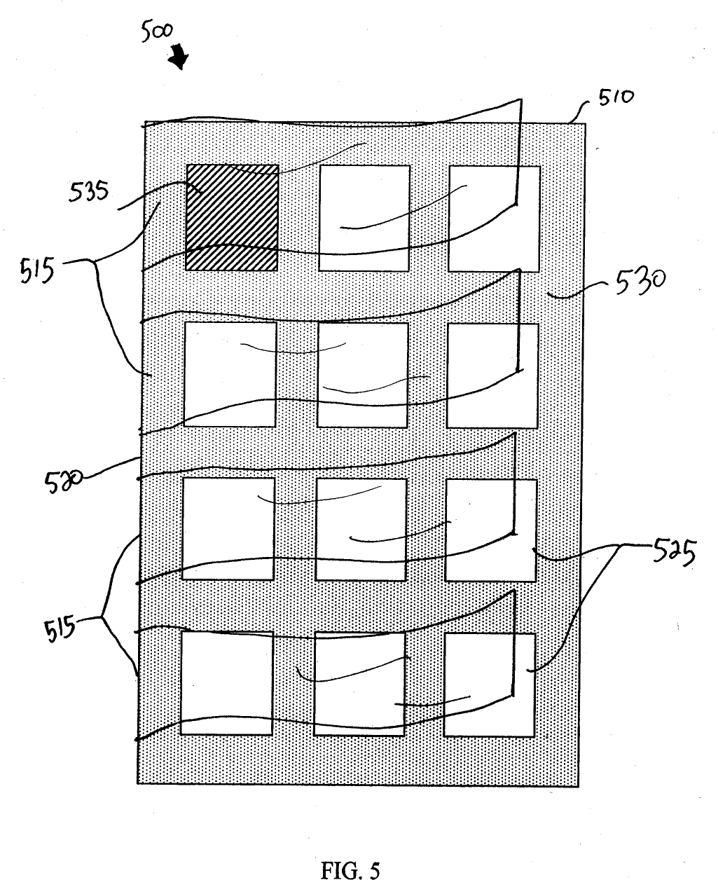

A further exemplary disposal system 500 is shown generally in FIG. 5. The system comprises disposal surface 510 and a plurality of covering layers 515, which are flexibly attached to the disposal surface at edge 520. Areas 525 are provided for placement of used transdermal delivery forms. Adhesive 530 is disposed on some or all of the portion of the disposal surface 510 which is between and around areas 525. As described above, the transdermal delivery form may have adhesive on part or all of its skin-facing surface. Alternatively, the transdermal delivery system has no adhesive. A transdermal delivery form 535 is shown placed in an area 525 of disposal surface 510. Once one or more transdermal delivery forms have been placed on the disposal surface 510, the covering layers 515 can be positioned over and contacted with the disposal surface, such that the adhesive 530 secures the covering layers in place across a given row of used transdermal delivery forms. The arrangement of a plurality of covering layers 515, each of which are positionable over a given row of areas 525, allows for successive rows to be sealed by a covering layer as they are filled with used transdermal delivery forms.

It is understood that the some or all of the surface of the covering layer which is intended to contact the disposal surface carrying the used transdermal delivery forms can be coated with one or more adhesives as discussed above. This adhesive can be present on the covering layer surface in addition to, or in place of, the adhesive on the disposal surface.

The transdermal delivery form may be fixed to the disposal system surface such that that it cannot be removed without at least partially destroying the matrix containing the controlled substance, or such that the matrix containing the opioid is rendered at least partially inaccessible to extraction methods. As used herein, “at least partially inaccessible to extraction methods” means that the ability of the controlled substance to be chemically or physically removed from the disposal surface is hindered or blocked; i.e., the amount of opioid that can be removed is less than that which can be removed from a comparable used transdermal delivery form that has not been fixed to the disposal surface.

A method of disposing of one or more transdermal delivery forms containing controlled substances is also provided. In the practice of this aspect of the method, a disposal system as discussed above is provided. One or more used transdermal delivery forms are placed on the at least one disposal surface in a defined or undefined area. The used transdermal delivery device may be placed on a defined area which is marked, so that the user can track or otherwise account for the used delivery devices. For example, the markings identifying each defined area on the disposal surface can correspond to a given transdermal delivery form.

Once the transdermal delivery form is placed on the disposal surface, the user can then apply pressure or heat, or otherwise fix the delivery form in place with a fastening device as discussed above. The heat, pressure or fastening device can be applied manually, or can be applied with a machine adapted for that purpose. For example, a disposal surface on which one or more used transdermal forms have been placed can be fed through a machine comprising at least two rollers, which are spaced so that the disposal surface and transdermal forms are compressed together. The machine can optionally comprise a heating element that heats the disposal surface and transdermal forms and, for example, melts or activates an adhesive or other thermosetting substance to fix the used delivery devices on the disposal surface.

Once the transdermal forms have been fixed to the disposal surface, the disposal system can be discarded. The ability of the opioid to be extracted or otherwise recovered from the disposal system is reduced or eliminated. Thus, the disposal system also allows for a method of reducing or preventing the diversion of opioids by individuals intending to provide residual opioids in the used transdermal delivery forms to opioid addicts.

A method of producing a disposal system for one or more transdermal delivery forms containing controlled substances is also provided. The method comprises the formation of at least one surface for receiving the transdermal delivery form, which can comprise one or more structures for fixing a transdermal delivery form to the at least one surface. Formation of suitable disposal surfaces can be accomplished by techniques within the skill in the art, as discussed above. For example, disposal surfaces can be fabricated from plastic or rubber materials by techniques such as blow-molding, injection molding, stamping and the like. The covering layer, if present, can also be fabricated (for example from plastic or rubber materials) by techniques well-known in the art.

The adhesives for use on the disposal surface or covering layer, in particular the pressure sensitive adhesives, can be prepared and coated onto the disposal surface or covering layer using a variety of standard techniques. For example, the pressure sensitive adhesives can be polymers that are dispersed in solvent or water, and then coated onto the disposal surface or covering layer. If a solvent-borne or water-borne pressure sensitive adhesive composition is employed, then the adhesive layer may undergo a drying step to remove some or substantially all of the carrier liquid. The adhesive may be cured using an energy source (e.g., heat, UV radiation, electron beam, and the like). Alternatively, adhesives may be applied without dispersal in a solvent or water using a variety of methods, such as, for example, melting or extruding the adhesive onto the disposal surface or covering layer. Techniques and apparatuses for applying adhesive to the disposal surface or covering layer in a pre-selected pattern are also well-known in the art.

Transdermal delivery forms which contain opioids can be manufactured according to standard techniques, as discussed above. The transdermal delivery forms can also be manufactured to contain substances which reduce the oral, intranasal or parenteral effectiveness of opioids extracted from the delivery forms, according to standard techniques. The presence of such substances can further constrain attempts at diversion of residual opioids contained in used transdermal delivery forms. Examples of suitable substances include, but are not limited to, opioid antagonists such as naloxone, naltrexone, nalorphine, diprenorphine, levallorphan, pentazocine, metazocine, cyclazocine, etazocine, N-cyclopropylmethyl-7,8-dihydro-14-hydroxynormorphinone, and 21-cyclopropyl z,-(1-hydroxy-1-methylethyl)-6,14-endo-ethano-tetrahydrooripavine(diphenorphine), the pharmaceutically acceptable acid addition salts thereof, and any combinations of these.

The amount of opioid antagonist used in the transdermal dosage forms can be readily determined by one of ordinary skill in the art. For example, US Patent Publication 2003/0004177, the entire disclosure of which is herein incorporated by reference, indicates that about 0.2 to about 0.4 mg naloxone is needed to antagonize the opioid effect in parenteral or intranasal administration. However, because of the reduced efficacy of naloxone when taken orally, substantially greater amounts are needed to prevent oral abuse when naloxone is used as the antagonist. Accordingly, at least about 0.1 mg, for example at least 1.0 mg, at least about 5.0 mg, or at least about 20 mg per 100 mg opioid can be used to antagonize the effect of extracted opioids in oral administration.

Other amounts of naloxone used to antagonize the effect of certain opioids are possible. For example, the combination of pentazocine and naloxone has been utilized in tablets available commercially available in the US as Talwin®Nx from Sanofi-Winthrop. Talwin®Nx contains pentazocine hydrochloride equivalent to 50 mg base and naloxone hydrochloride equivalent to 0.5 mg base. A fixed combination of buprenorphine and naloxone was introduced in 1991 in New Zealand as Temgesic®Nx by Reckitt & Colman. A fixed combination therapy comprising tilidine (50 mg) and naloxone (4 mg) has been available in Germany for the management of severe pain since 1978 (Valoron®N; Goedecke). U.S. Pat. No. 4,457,933, the entire disclosure of which is herein incorporated by reference, describes a method for decreasing both the oral and parenteral abuse potential of oxycodone, propoxyphene or pentazocine by combining an analgesic dose of the opioid with naloxone in a ratio of 2.5-5:1 parts by weight. Pentazocine-naloxone compositions having a ratio of 16-50:1 parts by weight were preferred. U.S. Pat. No. 4,582,835, the entire disclosure of which is herein incorporated by reference, describes compositions of buprenorphine with naloxone in ratios of naloxone to buprenorphine from 1:3 to 1:1 for parenteral administration, and from 1:2 to 2:1 for sublingual administration. Lesser amounts of opioid antagonists that have greater oral bioavailability than naloxone can be used.

Oral or intranasal administration of opioids can also be curtailed by including irritants, such as capsaicin, in the transdermal delivery form. Capsaicin is the natural ingredient found in chili peppers and other species of the Capsicum genus, and is known to be an irritant, particularly to mucosal membranes. Derivatives and analogs of capsaicin (i.e., nordihydiocapsaicin and dihydrocapsaicin) are known generally as “capsaicinoids,” and are also suitable irritants for inclusion in the transdermal delivery forms. Although some studies suggest that capsaicin can have a synergistic effect with certain analgesics, certain amounts of capsaicin included in transdermal delivery forms have been found deter the intranasal, oral or intravenous use of opioid extracts made from such delivery forms.

The amount of capsaicin or capsaicinoids that can be included in transdermal delivery devices to deter abuse of an opioid extract is that amount which produces irritating effects on the user, such as coughing, sneezing, burning, and/or pain. The pain and discomfort associated with capsaicin may endure for minutes and potentially hours. These irritating effects are generally more noticeable and have a more direct onset when the opioid extract is taken orally or intranasally. However, the irritating effects can also be felt at or near the site of a parenteral injection of the opioid extract, if a portion of the extract contacts nearby skin or muscle tissue.

Suitable amounts of capsaicin or capsaicinoids can be from about 75 micrograms to about 250 micrograms total in the transdermal delivery form, for example about 125 micrograms or less. Greater or lesser amounts of capsaicin or capsaicinoids for use in the transdermal delivery form may be used. Guidance regarding the amount of capsaicin or capsaicinoids that can be used, and a description of techniques for producing transdermal delivery forms comprising such irritants to prevent oral, intranasal or parenteral abuse, are described in US Patent Publication 2003/0064122, the entire disclosure of which is herein incorporated by reference (see, in particular, Table 1 therein).

An emulsifier can be included in the transdermal delivery form in order to make the capsaicin or capsaicinoid more soluble in aqueous extraction media. Suitable emulsifiers include mono and diglycerides, laureates such as sodium lauryl sulfate, oleates, glycols, or docusate sodium. Suitable amounts of emulsifier to be included in the transdermal delivery form are as described in Table 2 of US 2003/0064122, supra.

A variety of modifications to the aspects described will be apparent to those skilled in the art from the disclosure provided herein. Thus, the invention may be embodied in other specific forms without departing from the spirit or essential attributes thereof and, accordingly, reference should be made to the appended claims, rather than to the foregoing specification, as indicating the scope of the invention.

I claim:

1. A disposal system for one or more used transdermal delivery forms containing controlled substances, comprising at least one disposal surface for receiving the one or more used transdermal delivery forms and one or more structures for fixing the transdermal delivery form to the disposal surface.

2. The disposal system of claim 1, wherein the one or more structures for fixing the used transdermal delivery form to the disposal surface are located on or attached to the at least one disposal surface.

3. The disposal system of claim 1, wherein the one or more used transdermal delivery forms comprise a reservoir for the controlled substance.

4. The disposal system of claim 1, wherein the one or more structures for permanently fixing the transdermal delivery form to the disposal surface are located on or attached to the one or more used transdermal delivery forms.

5. The disposal system of claim 1, wherein the one or more used transdermal delivery forms comprise an opioid.

6. The disposal system of claim 5, wherein the opioid is selected from the group consisting of alfentanil, buprenorphine, butorphanol, codeine, dezocine, dihydrocodeine, fentanyl, fentanyl congeners, hydrocodone, hydromorphone, levorphanol, meperidine (pethidine), methadone, morphine, nalbuphine, oxycodone, oxymorphone, pentazocine, propiram, propoxyphene, tilidine, tramadol, the pharmaceutically acceptable acid addition salts thereof, and any combinations of these.

7. The disposal system of claim 6, wherein the fentanyl congeners are at least one selected from the group consisting of sufentanil, alfentanil, lofentanil, carfentanil, remifentanil, trefentanil and mirfentanil.

8. The disposal system of claim 1, wherein the one or more used transdermal delivery forms further comprise a salicylate, acetaminophen, phenylpropanolamine, phenylephrine, chlorpheniramine, caffeine or guaifenesin.

9. The disposal system of claim 1, wherein the one or more used transdermal delivery forms comprise adhesive disposed on substantially the entire surface intended to contact the skin or disposed around substantially the entire perimeter of the surface intended to contact the skin.

10. The disposal system of claim 1, wherein the at least one disposal surface comprises one or more defined areas for receiving the one or more used transdermal delivery forms.

11. The disposal system of claim 10, wherein the defined areas are identified by markings which correspond with the one or more transdermal delivery forms.

12. The disposal system of claim 1, wherein the one or more structures for fixing the transdermal delivery form to the disposal surface comprise one or more adhesives disposed on the at least one disposal surface.

13. The disposal system of claim 1, wherein the one or more structures for fixing the transdermal delivery form to the disposal surface comprise a fastening device.

14. The disposal system of claim 1, wherein the one or more structures for fixing the transdermal delivery form to the disposal surface comprise at least one covering layer.

15. The disposal system of claim 14, wherein the at least one covering layer comprises one or more adhesives.

16. The disposal system of claim 1, wherein the used transdermal delivery forms comprise at least one substance which reduce the oral, intranasal or parenteral effectiveness of controlled substances extracted from the used transdermal delivery forms.

17. The disposal system of claim 16, wherein the substance is an opioid antagonist, capsaicin or a capsaicinoid.

18. A method of disposing of one or more used transdermal delivery forms containing controlled substances comprising:
(1) providing the disposal system of claim 1;
(2) placing one or more used transdermal delivery forms on the at least one disposal surface; and
(3) fixing the one or more used transdermal delivery forms to the at least one disposal surface.

19. A method to reduce or prevent diversion of controlled substances contained in one or more used transdermal delivery forms, comprising:
(1) providing the disposal system of claim 1;
(2) placing one or more used transdermal delivery forms on the at least one disposal surface; and
(3) fixing the one or more used transdermal delivery forms to the at least one disposal surface.

20. A method of producing a disposal system for one or more used transdermal delivery forms containing a controlled substance comprising:
forming at least one disposal surface for receiving the one or more used transdermal delivery forms; and
associating the one or more structures for fixing the one or more used transdermal delivery forms to the at least one disposal surface with the at least one disposal surface.
